(19) United States
(12) Patent Application Publication
(10) Pub. No.: US 2006/0013723 A1
(43) Pub. Date: Jan. 19, 2006

(54) Wine-based disinfectant

(76) Inventors: Mark A. Daeschel, Philomath, OR (US); Jessica R. Just, Petaluma, CA (US); Joy Waite, Corvallis, OR (US)

Correspondence Address: KLARQUIST SPARKMAN, LLP, 121 SW SALMON STREET, SUITE 1600, PORTLAND, OR, 97204, US

(21) Appl. No.: 10/535,515

(22) PCT Filed: Nov. 26, 2003
(86) PCT No.: PCT/US03/37993

§ 371 Date: May 18, 2005

Related U.S. Application Data
(60) Provisional application No. 60/429,927, filed on Nov. 27, 2002.

(51) Int. Cl. A61L 2/04

(52) U.S. Cl. 422/1

(57) ABSTRACT

The present disclosure relates to compositions for use as disinfectants. The compositions disclosed herein include wine, salt, and sulfur dioxide. In one example, the composition includes a waste wine, at least about 1.5% w/v salt, at least about 0.5% alcohol, and at least about 30 ppm sulfur dioxide. Methods are provided for using such compositions to disinfect various objects.

### CROSS REFERENCE TO RELATED APPLICATION

This application claims the benefit of U.S. Provisional Application No. 60/429,927, filed Nov. 27, 2002, herein incorporated by reference in its entirety.

### FIELD

The present disclosure relates to compositions useful as disinfectants, and methods of their use.

### BACKGROUND

Food-borne illness affects approximately 76 million people in the United States each year (Tierney et al., 2002. Dairy, Food and Environmental Sanitation 22:658-66). The food industry, along with the Centers for Disease Control (CDC), has worked diligently to control the conditions surrounding processed foods through plans such as Hazard Analysis Critical Control Points. Due to the increased use of these control measures, the CDC reported a 23 percent drop in bacterial food-borne illnesses between 1996 and 2002.

Although significant progress has been made at the industry level, it is important to further reduce other sources of food-borne illness. Although restaurant outbreaks of food-borne illness are highly publicized, studies indicate that between 19 and 80 percent of food-borne infections arise in the home (Tierney et al., 2002. Dairy, Food and Environmental Sanitation 22:658-66 and Zhao et al., 1998. J. Food Protection 61:960-3). The kitchen is responsible for harboring and transferring most bacteria in the house, such as pathogenic bacteria, including Staphylococcus aureus, pseudomonads, and enterobacteria (Scott et al. 1982. J. Hygiene 89:279-93). The sink area, including sponges used for washing dishes and wiping down surfaces, can spread the contamination onto ready-to-eat foods and throughout the kitchen (Josephson et al. 1997. J. Appl. Microbiol. 83:737-50).

The majority of commercial disinfectants are either hypochlorite, ammonia, iodine, or phenolic based, each of which can reduce the contamination of microorganisms in the household environment (Scott et al., 1984. J. Hygiene 92:193-203). However, phenolic disinfectants are not ideal, especially when used on dry surfaces such as worktops and cutting boards (Scott et al., 1984. J. Hygiene 92:193-203). Although hypochlorite is an effective disinfectant, evidence indicates that some species of microorganisms are more resistant to these products, such as Escherichia coli, Klebsiella species and Pseudomonas aeruginosa (Hui et al. 1994. Foodborne Disease Handbook: Diseases Caused by Bacteria. Vol 1. New York: Marcel Dekker, Inc. 613 p.). Phenolic disinfectants may also cause tainting of food (Scott et al., 1984. J. Hygiene 92:193-203). Furthermore, the current consumer trend is a desire for more natural and environmentally friendly products that lack the potential of food tainting and odor.

Therefore, there is a need for a disinfectant that is environmentally friendly.

### SUMMARY

Disclosed herein are compositions that include wine, salt, and sulfur dioxide (SO₂). The wine can come from any source, such as a waste wine, for example a wine product produced from vineyard cluster thinnings, pomace, or a combination thereof The disclosed compositions can be used as a disinfectant, for example to disinfect household surfaces or food products. Using both a liquid suspension test and a surface carrier test, the inventors have determined that compositions have disinfectant properties. In addition, sulfur dioxide is shown herein to enhance the anti-microbial activity of wine. In a particular example, the composition includes waste wine, at least about 1.5% w/v salt, and at least about 30 ppm of SO₂.

Methods of using the disclosed disinfectant compositions are also disclosed.

### BRIEF DESCRIPTION OF THE DRAWINGS

FIGS. 1 and 2 are graphs showing suspension results for (1) K. pneumoniae and (2) P. aeruginosa.

FIGS. 3 and 4 are graphs showing suspension results for (3) S. aureus 609 and (4) S. aureus 649.

FIG. 5 is a graph showing surface carrier results for K. pneumoniae.

### DETAILED DESCRIPTION OF SEVERAL EMBODIMENTS

The following explanations are provided to better describe the present disclosure and to guide those of ordinary skill in the art in the practice of the present disclosure. As used herein, the singular forms “a” or “an”, or “the” include plural references unless the context clearly dictates otherwise. For example, reference to “a disinfectant” includes one or a plurality of such disinfectants, and reference to “the surface” includes reference to one or more surfaces and equivalents thereof known to those skilled in the art, and so forth. The term “or” refers to a single element of stated alternative elements or a combination of two or more elements, unless the context clearly indicates otherwise. For example, the phrase “a cluster thinnings wine or a pomace wine” refers to cluster thinnings wine, pomace wine, or a combination of both cluster thinnings wine and pomace wine. As used herein, “comprises” means “includes.” Thus, “comprising a waste wine and sulfur dioxide,” means “including a waste wine and sulfur dioxide,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

Agent: Any substance, including but not limited to a chemical compound.

Animal: A multi-cellular organism characterized by voluntary movement. Examples include, but are not limited to, mammals and birds, such as cows, pigs, lambs, chickens, and turkeys, as well a fish and shellfish.

Cluster thinnings: Grapes harvested earlier than the main harvest, for example to hasten the maturity and improve the quality of the remaining grapes. Generally, cluster thinnings are not suitable for making wine for consumption. In one example, cluster thinning grapes have a lower sugar content and higher acid content than main harvest grapes. Cluster thinnings wine is wine produced by fermenting cluster thinnings.

Disinfectant or anti-microbial: A composition which includes one or more agents that, alone or in combination with other agents, destroy, neutralize, decrease the growth of, or decrease the number of, one or more microbes by a desired amount.

Disinfectants reduce the number of microbes present in a liquid suspension by at least about 90% after one minute, for example by at least about 95%, for example by at least about 99% or even at least about 99.99% after one minute. In a particular example, a composition is a disinfectant if it can reduce the number of microbes present on a surface by at least about 50% after 15 minutes, for example by at least about 70%, at least about 80%, at least about 90%, at least about 95%, at least about 97% or even at least about 98% after 15 minutes. In yet another or additional example, a composition is said to be a disinfectant if it can reduce the number of microbes present on a surface by at least about 95% after 30 minutes, for example by at least about 98%, at least about 99%, or even at least about 99.5% after 30 minutes.

In a particular example, a composition is said to be a disinfectant if it can reduce the number of Klebsiella pneumoniae, S. aureus, Pseudomonas aeruginosa, or combinations thereof, present in a liquid suspension by at least about 90% after one minute, for example by at least about 99% or even at least about 99.99% after one minute. In another or additional particular example, a composition is said to be a disinfectant if it can reduce the number of K pneumoniae bacteria present on a surface by at least about 50% after 15 minutes, for example by at least about 90%, at least about 95% or even at least about 97% after 15 minutes.

Disinfectants can be applied to the surface of objects that may have microbes present on them.

Microbe: An infectious agent that causes disease. Non-limiting examples of microbes include bacteria, viruses, and fungi. Particular examples include E. coli, K. pneumoniae, P. aeruginosa, and S. aureus.

Pomace: The pulpy material (such as skins and seeds) remaining after the juice has been pressed from fruit, such as grapes. In one example, pomace includes the grape skins and seeds left over after pressing the grapes in the production of wine. Generally, pomace contains the same amount of sugar and acid as the juice extracted from it. Pomace wine is wine produced by fermenting pomace.

Salted wine: A wine or wine product not for beverage use produced in accordance with the provisions of 27 C.F.R. § 24.215 and having not less than 1.5 grams of salt per 100 milliliter of wine (12.5 pounds of salt/100 gallons of wine).

Surface: The outer, or top part or layer, of something, such as an inanimate object or food product. Surfaces can include any material, such as Formica, plastic, stone, marble, steel, wood, and glass. Examples of surfaces include, but are not limited to surfaces found in kitchens and bathrooms, such as counter-tops, toilets, cutting boards, trash cans, sinks, sponges, floors, and bathtubs. In one example, the surface is the surface of an animal carcass, for example a chicken, cow, or pig carcass. In a particular example, the surface is the surface of a fruit or vegetable.

Viable: Capable of working, functioning, or developing adequately. For example, the ability of a microbe to continue surviving, further develop, reproduce, or a combination thereof.

Waste wine: Any waste product or surplus (or a combination thereof) generated during the production of wine. Exemplary waste products include, but are not limited to, cluster thinnings, pomace, waste wine, and combinations thereof During the normal fermentative and industrial processes involved in the production of wine, problems can arise, which result in the production of waste wine. For example, when the volatile acidity of wine exceeds exceed 0.140 g/100 ml acetic acid and lactic acid, it cannot be sold to the public, and renders some batches of wine as waste products (Zoecklein et al., 1990. Production Wine Analysis. New York: Van Nostrand Reinhold. 475 p.). In another example, a batch of wine may not meet buyer specifications for quality and cannot be sold, thus generating waste wine.

White wine: Any wine of a clear, transparent color, such as pale yellowish to bordering on white, as distinguished from wines of a deep red color. White wine can be made from white grapes, or from red grapes with skins removed before fermentation. Particular examples of white wine include, but are not limited to chardonnay, Sauvignon blanc, Riesling, pinot blanc, Gewurztrariner, and pinot gris, in contrast to red wines such as port, zinfandel, pinot noir, and Burgundy.

Wine: Includes every kind (class and type) of product produced from fermentation of grapes, other fruit (including berries), or other suitable agricultural products and containing at least about 0.5% alcohol by volume, but not more than about 24% of alcohol by volume, for example about 8%-24% alcohol by volume, such as about 10-24% alcohol by volume, such as about 10-15% alcohol by volume. The term includes all imitation or artificial wine and compounds sold as wine. In some examples, it includes wine produced by fermenting cluster thinnings, pomace, or a combination thereof.

Wine can have at least about 0.6% alcohol by volume, such as at least about 1% alcohol by volume, at least about 5% alcohol by volume, at least about 8% alcohol by volume, at least about 10% alcohol by volume, at least about 12% alcohol by volume, or at least about 14% alcohol by volume.

### Disinfectant Compositions

Wine results from the fermentation primarily of grapes, which produces a product containing alcohol and organic acids, such as tartaric, malic, and lactic acid. Wine producers have few options for waste wine and surplus wine produced during the process of making wine. In addition, wine producers have little use for vineyard cluster thinnings, which are typically discarded and left to decompose on the vineyard floor. Similarly, pomace (typically skin and seeds) generated from pressing the grapes at harvest is usually composted with other materials and deposited on the vineyard floor or otherwise discarded.

The present disclosure provides new disinfectant compositions that include waste products generated during the production of wine that would otherwise have little financial value to the wine producer. In one example, the disclosed compositions include waste wine, surplus wine, or a combination thereof, supplemented with salt and sulfur dioxide, to increase the disinfectant properties of the composition, and provide an environmentally conscious alternative to chlorine and iodine based disinfectants.

The disinfectant compositions disclosed herein include wine, salt, and SO₂. In one example, the wine is a waste wine, for example a waste white wine, or a surplus wine. The disclosed compositions include waste wine, at least about 1.5% salt (such as NaCl) w/v, and at least about 30 ppm SO₂.

Any salt can be used in the disclosed compositions, such as NaCl or KCl, and is present at a concentration of at least about 1.5% w/v in the composition, but no more than about 10% salt. Ranges of salt that can be present in the disclosed compositions are about 1.5% -about 5% w/v salt. For example, the disclosed compositions can include at least about 2% w/v salt, at least about 3% w/v salt, or at least about 5% w/v salt.

The disclosed compositions include at least about 30 parts per million (ppm) SO₂ but no more than about 750 ppm SO₂. Ranges of SO₂ that can be present in the disclosed compositions are about 30-500 ppm SO₂, such as about 30-350 ppm SO₂, such as about 30-220 ppm SO₂, such as about 50-200 ppm SO₂. For example, the disclosed compositions can include at least about 50 ppm SO₂, at least about 100 ppm, at least about 125 ppm, at least about 150 ppm, at least about 175 ppm, at least about 200 ppm, at least about 250 ppm, at least about 300 ppm, at least about 350 ppm, at least about 500 ppm or at least about 700 ppm SO₂.

The concentration of alcohol in the disclosed compositions is at least 0.5% alcohol by volume, but no more than about 24% by volume. In some examples, the concentration of alcohol is at least about 1% by volume, such as at least about 5% by volume, at least about 8% by volume, at least about 10% by volume, at least about 12% by volume, or at least about 15% alcohol by volume. Ranges of alcohol that can be present in the disclosed compositions include about 0.5% -about 24% by volume, about 5%-about 15% by volume, or about 5% -about 12% by volume.

The disclosed compositions have a pH of about 2.5-4.0, such as about 2.6-3.8. In some examples, the pH is at least about pH 2.6, for example at least about pH 3, for example at least about pH 3.5, for example at least about pH 4.

The disclosed compositions can include additional agents. The compositions can include a titratable acidity as tartartic acid of about 5-about 35 g/L, such as about 5-about 30 g/L, or about 5-about 15 g/L. In some examples, a titratable acidity as tartartic acid of is at least about 5 g/L, for example at least about 10 g/L, for example at least about 12 g/L, at least about 15 g/L, at least about 20 g/L, at least about 30 g/L, or even at least about 35 g/L. The compositions can also include a volatile acidity as acetic acid of 0-10 g/L, such as 0.1-10 g/L. In some examples, the volatile acidity as acetic acid is at least about 0.1 g/L, for example at least about 0.2 g/L, at least about 0.5 g/L, at least about 1 g/L, at least about 2 g/L, at least about 5 g/L, or at least about 10 g/L.

The disclosed compositions can also include one or more coloring or fragrance compounds.

The disclosed compositions reduce the number of viable microbes by a desired amount. Incubating the disclosed compositions with a liquid suspension reduces the number of viable microbes by at least about 90% after 1 minute, for example at least about 95%, at least about 99%, or even at least about 99.99% after one minute. In addition, incubating the disclosed composition with a liquid suspension reduces the number of viable microbes by at least about 70% after 20 minutes, for example by at least about 80%, at least about 85%, at least about 90%, at least about 95%, at least about 98%, or even at least about 99.9%.

Contacting the disclosed composition with a surface reduces the number of viable microbes by at least about 50% after 15 minutes, for example by at least about 70%, at least about 90%, at least about 95%, or even at least about 97% after 15 minutes. Contacting the disclosed composition with a surface reduces the number of viable microbes by at least about 90% after 30 minutes, for example by at least about 95%, at least about 97%, at least about 98%, at least about 99% or even at least about 99.5% after 30 minutes.

### Methods of Disinfection

Methods of disinfecting using the disclosed compositions are also provided. For example, the method can include contacting an object, such as the surface of the object, with the disclosed compositions. Objects and surfaces that can be disinfected include but are not limited to, those present in a bathroom or kitchen, such as a countertop, door, sink, cutting board, toilet, bathtub, shower, or floor. In another example, the object is a food product, such as an animal carcass, for example a chicken, cow, or pig carcass, which is contacted with the disclosed compositions to disinfect the carcass, for example by reducing the number of viable microbes present on the carcass. Other exemplary food products include fruit and vegetables, which can be contacted with the disclosed compositions to disinfect the fruit and vegetables, for example by reducing the number of viable microbes present on the fruit and vegetables.

These and additional features are further explained by the following non-limiting examples.

### EXAMPLE 1

### Suspension Test

This example describes methods that were used to test the growth of model microbes: Klebsiella pneumoniae, Pseudomonas aeruginosa and Staphylococcus aureus in various compositions, by using a liquid suspension test. The suspension test can be used as a rapid method to determine the relative effectiveness of a disinfectant composition against several microbes at different time intervals.

K. pneumoniae and P. aeruginosa were cultivated and enumerated with tryptic soy broth/agar (Becton Dickinson and Company, Sparks, Md.). S. aureus strains were cultivated and enumerated with brain heart infusion broth/agar (Becton Dickinson and Company). Enumeration was facilitated with an automated spiral plater (Autoplate 4000, Spiral Biotech, Norwood, Mass.) and performed in duplicate.

Chardonnay grapes were obtained from Oregon State University vineyards. Grapes were crushed, treated with 50 ppm sulfur dioxide and frozen until use. Subsequently, the thawed grapes were pressed off the skins to obtain juice. A portion of each juice was frozen and stored at −20° C. until further use. The remaining Chardonnay juices were fermented to completion with the commercial wine yeast DV10 (Scott Laboratories, Petaluma, Calif.) according to the manufacturer's instructions. In some experiments, Chardonnay wine was modified by adding potassium metabisulfite (resulting in a concentrations of 100 or 200 ppm sulfur dioxide), and 1.5 % w/v NaCl.

The disclosed wine-based compositions were compared to Seventh Generation Kitchen Cleaner (Seventh Generation, Inc., Burlington, Vt.). This product contains hydrogen peroxide as the active ingredient and is advertised as a natural environmentally-friendly alternative to mainstream kitchen disinfectants.

Overnight bacterial test culture (1 ml) was transferred to 99-mL Butterfield's phosphate buffer (Dilu-Lok Plus, Hardy Diagnostics, Santa Maria, Calif.). This served as the inoculum for the suspension test. The inoculum was diluted and plated to determine the number of bacterial cells. Each disinfectant formulation (6 ml) was added to a 17×100 mm sterile culture tube with closure (VWR International, Brisbane, Calif.). Inoculum (0.67 ml) was added to each disinfectant and the tube shaken for approximately one minute prior to each plating. K. pneumoniae and P. aeruginosa were enumerated at 1, 3, and 5 minutes while S. aureus strains were enumerated at 1, 3, 5, 7, and 9 minutes.

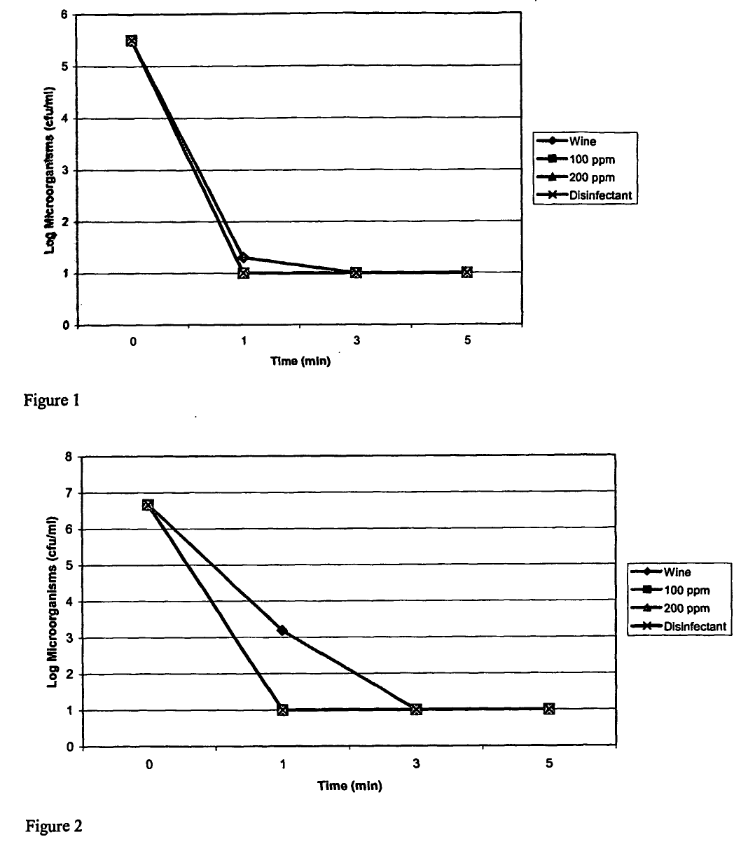

As shown in FIG. 1, the initial inoculum level for K. pneumoniae in liquid suspension was 5.50 log colony forming units (cfu)/ml. After one minute all of the treatments, except the unmodified wine, exhibited at least a 4.50 log reduction to the detection limit of 10 cells/ml. The unaltered original chardonnay wine displayed a 4.20 log reduction after only one minute of exposure time. After three minutes of exposure, the original chardonnay also reached the detection limit.

Similar results were obtained for P. aeruginosa (FIG. 2). All disinfectants except the original chardonnay achieved a reduction of 5.67 logs form the initial level of 6.67. The original wine was not as effective as the other treatments displaying a 3.48 log reduction after one minute, but reached the detection limit after 3 minutes of exposure.

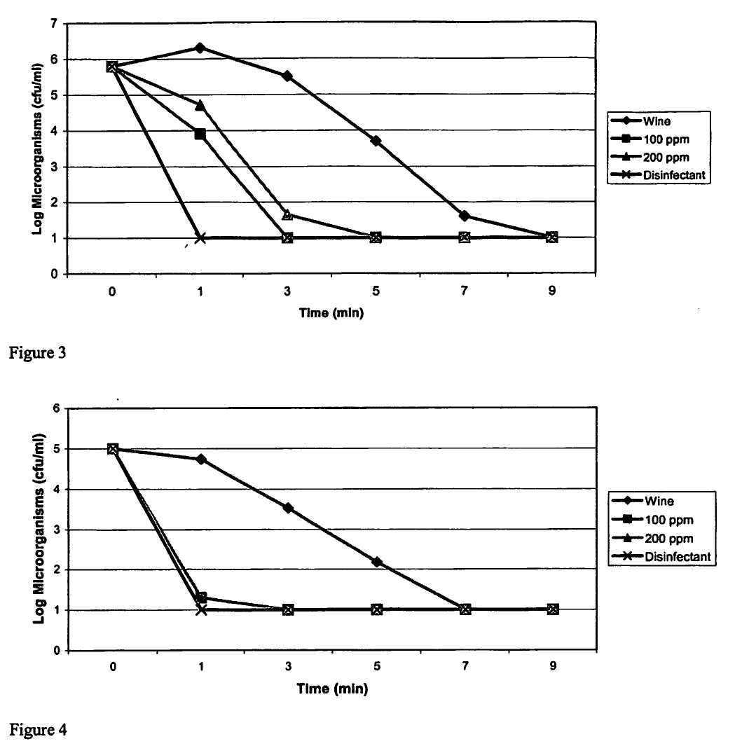

The suspension test results for S. aureus 609 are shown in FIG. 3. S. aureus is known for its resistance to disinfectants, and is further shown in FIG. 3. While the original inoculum level was 5.79 log cfu/ml, less than that of P. aeruginosa (FIG. 2), a longer exposure time was needed to achieve the same level of kill on a per cell basis. At three minutes of exposure and beyond, there is a trend downwards until after 9 minutes at which the detection limit was reached. Wine-based compositions which included 1.5% NaCl and 100 ppm SO₂ resulted in a 1.88 log reduction after 1 minute and then reached the detection limit of 10 cells/ml by 3 minutes of exposure time. In addition, wine-based compositions with the same amount of NaCl but containing 200 ppm SO₂, resulted in a 1.07 log reduction after 1 minute, and by 5 minutes, reached the detection limit. The commercial disinfectant inactivated S. aureus to the detection limit after one minute of exposure time.

Similar results were obtained for S. aureus 649 (FIG. 4). The unmodified wine was not as effective as the other formulations. The unmodified wine took 7 minutes before complete kill was achieved starting with an initial inoculum of 5.0 log cfu/ml. The other wine-based disinfectants showed a 3.7 log reduction after 1 minute and complete reduction after 3 minutes (FIG. 4). The commercial disinfectant showed a complete 4.0 log reduction after one minute.

In summary, all bacterial species tested were more rapidly destroyed in the wine-based composition supplemented with salt and SO₂, than the original wine. Although the original chardonnay had some disinfectant properties, this was likely due to the acids and alcohol present, since the original chardonnay had no salt and very low levels of SO₂.

### EXAMPLE 2

### Carrier Test

This example describes methods that were used to test the growth of K. pneumoniae and P. aeruginosa in various compositions, by using a carrier test. The carrier test was used to simulate a kitchen environment. A large percentage of households in the United States have countertops made of Formica, so this material was used as a model test surface. The discs were first sanded to allow the inoculum to more evenly spread over the surface.

Round 1.25-inch diameter sanded Formica-brand composite plastic discs were sterilized in 70% ethanol for approximately 10 minutes. Discs were aseptically transferred to a sterile 6-cell tissue culture plates (Falcon, Becton Dickinson Labware, Franklin Lakes, N.J.) and allowed to dry (approximately 30 minutes) in a Class II biological safety hood.

An overnight K. pneumoniae culture (1 ml) was mixed with 1 ml of 15% glycerol in a 17×100 mm sterile culture tube with closure (VWR International, Brisbane, Calif.). This inoculum was diluted and plated to determine the initial inoculum level. Inoculum (0.05 ml) was added to each disc and spread over the surface of the disc using a glass rod. The discs were allowed to dry for approximately 1.5 hours. Disinfectant or sterile deionized water (0.05 ml) was added to each disc and spread over the surface of the disc using a glass rod. Sterile water was used as a control to assess the impact of the drying steps.

At the appropriate time of exposure 0, 15, or 30 minutes, the discs were aseptically turned over (face down). Sterile glass beads (0.5 grams of 425-600 micron beads, Sigma-Aldrich Company, St. Louis, Mo.) and 0.95 ml of Butterfield's phosphate buffer (Dilu-Lok Plus, Hardy Diagnostics, Santa Maria, Calif.) was added to each well. The lid to the tissue culture plate was put on and the plate was shaken for 5 minutes at speed setting 3 using a bench top incubator shaker (Environ Shaker, Lab Line Designers and Manufacturers, Melrose Park, Ill.) to remove any bacteria from the Formica surface. The liquid was then removed from the well and enumerated for bacterial survivors.

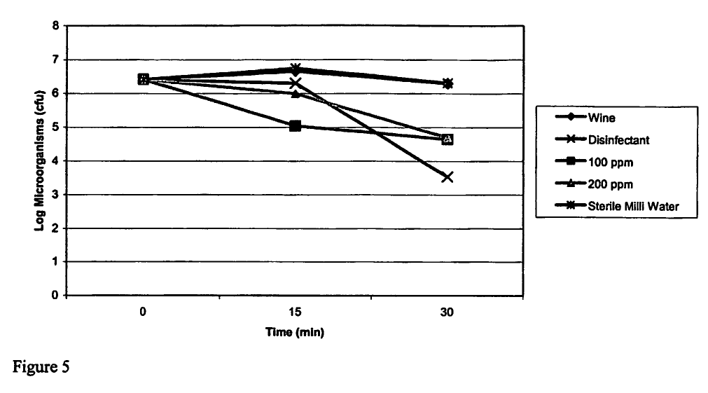

Using this method, the number of bacteria killed due to drying for 1.5 hours was determined. The original inoculum was 7.77 log cfu/disc, and after drying, this was reduced to an average of 6.41 cfu/disc, which is the value at time 0 (FIG. 5). As shown in FIG. 5, both sterile deionized water and the original wine had little effect on the viability the bacteria on a surface.

After 15 minutes of treatment time, the wine with salt and 100 ppm SO₂ achieved a log reduction of 1.37. After 30 minutes the commercial disinfectant achieved a log reduction of 2.82. While the commercial disinfectant required 30 minutes to provide a significant reduction, the supplemented wine showed significant reduction after 15 minutes (FIG. 5). Although no treatment achieved a complete kill after 30 minutes of exposure time, disinfectants in general tend to be less effective on surfaces as compared to a liquid environment (Aarnisalo et al., 2000. J. Food Safety, 20:237-50).

In summary, the results disclosed herein demonstrate that wine supplemented with salt and additional sulfur dioxide can exert significant antimicrobial activity against undesirable bacteria

### EXAMPLE 3

### Cluster Thinnings Wine as a Disinfectant

This example describes methods used demonstrate that wine produced from cluster thinnings can be used as a disinfectant.

Juice was pressed from winegrape cluster thinnings. The resulting juice contained 13.8% sugar, had a pH of about 2.75, and an acidity of about 1.83% tartaric acid. Commercial wine yeast was added to the juice, and the resulting composition allowed to ferment until all detectable sugar was gone. After fermentation with yeast, the composition contained about 6.0% alcohol by volume, 0% sugar, and about 1.88% tartaric acid.

To demonstrate the disinfectant properties of the wine produce from cluster thinnings, cells of Staphylocoocus aureus 710 and Escherichia coli O157:H7 716 were added to this mixture and the reduction of bacteria determined using the suspension test described in Example 1. Following 20 minutes of exposure to the cluster thinning wine composition, 87.83% of S. aureus were killed, and 99.97%. E. coli were killed.

### EXAMPLE 4

### Pomace Wine as a Disinfectant

This example describes methods used demonstrate that wine produced from pomace can be used as a disinfectant.

Fresh pomace from chardonnay and pinot grapes was added water to make an extract. Extracted juice from cluster thinnings and sugar were added to the pomace extract to achieve a concentration of 15 Brix alcohol. The resultant mixture was fermented with yeast as described in Example 3, and contained about 6.4% alcohol by volume, had a pH of about 2.79, 0% sugar and about 1.29% tartaric acid.

To demonstrate the disinfectant properties of the wine produced from pomace, cells of S. aureus 710 and E. coli O157:H7 716 were added to the pomace wine and the reduction of bacteria determined using the suspension test described in Example 1. Following a 20 minute exposure to pomace wine, S. aureus showed a 86.3% reduction, while E. coli showed a 99.91% reduction.

### EXAMPLE 5

### Enhancing Antimicrobial Activity

A commercial (no sulfite added) wine (Badger Mountain 2002 Chardonnay) was used as a base wine and combinations of pH, titratable acidity, sulfur dioxide and ethanol levels were adjusted to determine the effectiveness of these parameters alone and in combination on two pathogens S. aureus 710 and E. coli O157:H7. Both organisms were grown overnight in brain-heart infusion broth and plated on brain-heart infusion agar. Suspension tests were performed as described in Example 1, except that 100 μL of overnight culture served as the inoculum and was placed into 9.9 mL of the disinfectant to achieve an inoculum level of about 7.18 log cfu/ml, and the exposure time was increased to 20 minutes prior to plating.

The parameters were adjusted by adding the following: pH was decreased by adding 6N hydrochloric acid to achieve a pH of 3.25 or 3.00, titratable acidity was increased by adding 37% tartaric acid to increase the titratable acidity by 2 g/L and 4 g/L, sulfur dioxide was increased by adding solid potassium metabisulfite to increase sulfur dioxide levels by 50 ppm and 150 ppm, ethanol levels were increased by adding 95% ethanol to increase the ethanol content by 1.5% and 3.0%. pH adjustments were made first, followed by the remainder of the adjustments.

The base wine (no additions) reduced E. coli O157:H7 by 99.62% whereas S. aureus was reduced by 90.55%. However, as shown in Table 1, the base wine with no adjustments was the least effective disinfectant and all combinations increased the efficacy of the wine as a disinfectant. The harshest treatment (low pH, high titratable acidity, high sulfur dioxide, and high ethanol) reduced both organisms to the detection limit.

TABLE 1
Percent reduction in bacteria following exposure to disinfectants.

| Composition Conditions | S. aureus | E. coil |
|---|---|---|
| pH = 3.69 | 90.63% | 99.623% |
| titratable acidity = 6.75 g/L tartaric acid | | |
| sulfur dioxide = 30 ppm total | | |
| ethanol = 12.2% | | |
| (base wine) | | |
| pH = 3.05 | 99.9996% | 99.99997% |
| titratable acidity = 9.57 g/L tartaric acid | | |
| sulfur dioxide = 220 ppm total | | |
| ethanol = 14.4% | | |
| pH = 2.82 | 99.99995% | 100.00% |
| titratable acidity = 10.60 g/L tartaric acid | | |
| sulfur dioxide = 70 ppm total | | |
| ethanol = 12.4% | | |
| pH = 2.76 | 100.00% | 100.00% |
| titratable acidity = 12.82 g/L tartaric acid | | |
| sulfur dioxide = 158 ppm | | |
| ethanol = 14.2% | | |

In view of the many possible embodiments to which the principles of this disclosure may be applied, it should be recognized that the above examples should not be taken as a limitation on the scope of the invention. Rather, the scope of the invention is defined by the following claims.

We claim:

1. A composition comprising:
wine;
salt; and
sulfur dioxide (SO₂), wherein the composition is a disinfectant.

2. The composition of claim 1, wherein the wine is a waste wine, surplus wine, cluster thinnings wine, or pomace wine, or a combination thereof.

3. The composition of claim 1, wherein the salt is present at a concentration of at least about 1.5% w/v in the composition.

4. The composition of claim 1, wherein the sulfur dioxide is present at a concentration of at least about 30 parts per million (ppm) in the composition.

5. The composition of claim 1, wherein the sulfur dioxide is present at a concentration of about 30 ppm-about 350 ppm in the composition.

6. The composition of claim 1, wherein the composition further comprises a titratable acidity as tartartic acid of at least about 5 grams per liter.

7. The composition of claim 1, wherein the composition further comprises a titratable acidity as tartartic acid of about 5-about 30 grams per liter.

8. The composition of claim 1, wherein the composition further comprises a volatile acidity as acetic acid of at least about 0.3 gram per liter.

9. The composition of claim 1, wherein the composition further comprises a volatile acidity as acetic acid of about 0.3-about 10 grams per liter.

10. The composition of claim 1, wherein the composition has a pH of at least about 2.5.

11. The composition of claim 1, wherein the composition has a pH of about 2.5 to about 4.0.

12. The composition of claim 1, wherein the wine comprises at least about 5% alcohol by volume.

13. The composition of claim 1, wherein the composition reduces a number of viable microbes by at least about 90%.

14. A composition comprising:
a waste wine;
at least about 1.5% salt w/v; and
at least about 30 ppm sulfur dioxide, wherein the composition is a disinfectant and comprises no more than about 24% alcohol by volume.

15. A composition comprising:
a waste wine;
at least about 1.5% salt w/v; and
at least about 30 ppm sulfur dioxide, wherein the composition is a disinfectant and comprises at least about 5% alcohol by volume.

16. A composition comprising:
a waste wine;
about 1.5%-about 10% salt w/v; and
about 30 ppm-about 350 ppm sulfur dioxide, wherein the composition is a disinfectant and comprises about 5% to about 24% alcohol by volume.

17. A composition comprising:
a waste wine;
about 1.5%-about 10% salt w/v;
about 30 ppm-about 350 ppm sulfur dioxide, volatile acidity as acetic acid of about 0.3-about 10 grams per liter; and
titratable acidity as tartartic acid of about 5-about 30 grams per liter, wherein the composition is a disinfectant and has a pH of about 2.5 to about 4.0 and comprises about 5% to about 24% alcohol by volume.

18. A composition comprising:
a pomace wine;
at least about 1.5% salt w/v; and
at least about 30 ppm sulfur dioxide, wherein the composition is a disinfectant and comprises at least about 5% alcohol by volume.

19. A composition comprising:
a cluster thinning wine;
at least about 1.5% salt w/v; and
at least about 30 ppm sulfur dioxide, wherein the composition is a disinfectant and comprises at least about 5% alcohol by volume.

20. A method of disinfecting an object, comprising contacting the object with the composition of claim 1.

21. A method of disinfection, comprising:
contacting an object with the composition of claim 1;
allowing the composition of claim 1 to remain on the object for a period of time needed to reduce a number of viable microbes by a desired amount.

22. A method of disinfection, comprising:
contacting an object with the composition of claim 1;
allowing the composition of claim 1 to remain on the object for a period of time needed to reduce a number of viable S. aureus by at least about 99%.

23. A method of disinfection, comprising:
contacting an object with the composition of claim 1;
allowing the composition of claim 1 to remain on the object for a period of time needed to kill all detectable viable E. coli.

24. A method of disinfecting an animal carcass, comprising contacting the animal carcass with the composition of claim 1.
